(19) United States
(12) Patent Application Publication
(10) Pub. No.: US 2004/0142018 A1
(43) Pub. Date: Jul. 22, 2004

(54) Regulator for amount of body fat

(75) Inventors: Hiroyuki Takeuchi, Yokohama-Shi (JP); Jun Funabashi, Yokosuka-Shi (JP); Michio Kasai, Yokohama-Shi (JP); Masahiro Nakamura, Yokohama-Shi (JP); Hiroaki Tsuji, Yokohama-Shi (JP)

Correspondence Address: BURNS DOANE SWECKER & MATHIS L L P, POST OFFICE BOX 1404, ALEXANDRIA, VA, 22313-1404, US

(73) Assignee: The Nisshin OilliO, Ltd.

(21) Appl. No.: 10/753,412

(22) Filed: Jan. 9, 2004

Related U.S. Application Data
(63) Continuation of application No. PCT/JP02/06936, filed on Jul. 9, 2002.

(30) Foreign Application Priority Data
Jul. 16, 2001 (JP) 2001-214848

(51) Int. Cl. A61K 31/56; A61K 47/00; A61K 31/225; A61K 31/355; A61K 35/78

(52) U.S. Cl. 424/439; 514/171; 514/458; 424/776; 514/547

(57) ABSTRACT

The present invention provides a regulator for the amount of body fat containing a medium chain triglyceride, which can be used conveniently without any concern about the side effect and any restraint on the diet. 90% by mass or more of fatty acids constituting medium chain triglycerides are comprised of saturated fatty acids having 8 and 10 carbon atoms, the ratio by mass of the saturated fatty acids having 8 carbon atoms to the saturated fatty acids having 10 carbon atoms is 60:40 to 85:15, and the saturated fatty acids having 8 carbon atoms is present in an amount of 60 to 85% by mass of the total fatty acids bonded to the triglycerides at the 2-position. In addition, the present invention relates to a food product containing the above-mentioned regulator for the amount of body fat, which product is capable of regulating the body fat.

### BACKGROUND OF THE INVENTION

The present invention relates to a composition designed to regulate the amount of body fat, and more particularly to a regulator for the amount of body fat capable of safely and easily controlling the amount of body fat of the human body to an appropriate range, and a food product comprising the same.

It is well known that obesity resulting from overweight and excessive accumulation of the body fat tends to lead to various diseases, for example, metabolic disorder such as diabetes and hyperlipidemia and circulatory diseases such as hypertension and ischemic heart disease. The national nutrition survey conducted by the Ministry of Health and Welfare reports the result that one in seven adults is obese. As is obvious from such a result, the problem including overweight and obesity is considered to become one of the immediate concerns in Japan as well as in the Europe and the United States.

With respect to the body fat, the extremely low level of body fat is also known to have an adverse effect on the health. To be more specific, it has been found that the increase in the degree of obesity contributes to the high incidence of hypertension, diabetes and the like, and that the probability of development of those diseases also becomes high when the degree of obesity is low, that is, the amount of body fat is too small, as reported in “Himansho: Shindan, Chiryo, Shido no Tebiki [Obesity Handbook: Diagnosis, Treatment and

Instruction]” (Japan Society for the Study of Obesity, editorial board for a diagnosis handbook for obesity, pp. 11-12, Ishiyaku Publishers, Inc. 1993).

In light of the above, when focused on the body mass index (BMI), the most ideal BMI value is found to be 22, and the optimal weight (kg) is thus defined by formula of (height in meters)×(height in meters)×22, as shown in the above-mentioned reference, pp. 17-18.

Generally, obesity and overweight are simply caused by excessive caloric intake if there is no genetic factor or etiological factor (as described in the above reference, pp. 1-3). Most of the conventional slimming methods are therefore based on diets to restrict the caloric intake. Such dieting cannot be easily achieved because it is attended with mental stress. In addition, excessive dieting will lead to nutritional disturbance, and further cause the reduction of body fat more than required. In some cases, there is a risk of showing pathological symptoms, that is, the development of anorexia. Administration of pharmaceutical drugs is the other method for preventing the obesity. However, it is hard to say that administration of such drugs is easy and convenient for the individual to prevent the obesity. This is because consideration must be given not only to the efficacy of the drugs, but also to the side effects thereof.

To solve the above-mentioned problems, for example, low-calorie fats and oils (fat substitutes) have been developed. However, there is no fat substitute that can meet all the requirements in terms of safety, physical properties, cookability and taste. For example, U.S. Pat. No. 3,600,186 discloses that sucrose fatty acid esters can be used as low-calorie oils because the sucrose fatty acid esters can be excreted into the fecal matter without being absorbed by the digestive tract. Those sucrose fatty acid esters have been approved for use in salted snack foods in the United States, and in fact, potato chips cooked using the sucrose fatty acid esters are currently on the market. However, such products manufactured using the sucrose fatty acid esters are required to indicate the following statements: “This may cause abdominal spasm and loose stools” and “This inhibits the absorption of some fat-soluble vitamins”. The energy densities of protein and carbohydrate are not more than a half of the energy density of fat. As is known, it is possible to provide low-calorie fat substitutes by processing the protein and carbohydrate so as to have fat-like properties and taste (“Nutrition Reviews” Vol. 4, No. 4, pp.23-33, 1996). Such fat-substitutes make it possible to provide low-calorie ice cream and other desserts and so on. However, those fat substitutes have the disadvantages that the taste and flavor are still unsatisfactory and the heat resistance is too low to withstand high cooking temperatures. Further, those fat substitutes are not provided with the function of controlling the amount of body fat to an appropriate level.

Medium chain triglyceride has been utilized as solvents for oil-soluble fragrant substances and coloring substances for use in food, release oil and lubricant oil for food, raw materials for pharmaceutical products and the like because the medium chain triglyceride is substantially colorless and transparent and has high oxidation stability, low freezing point, low viscosity, and high solubility. Furthermore, in light of another advantages of the medium chain triglyceride, that is, excellent absorption and higher energy metabolism than saccharides, the medium chain triglyceride is also used as the raw material for enteral nutritional supplement with the aim of efficient supplementation of energy.

It is reported that accumulation of the body fat in the animals can be reduced by replacing the fat components contained in the animal feed with the medium chain triglyceride (Andrea et al., Life Sciences, Vol. 62, pp. 1203-1215, 1998). However, it has not been clarified that the medium chain triglyceride is provided with the function of controlling the amount of body fat. Also, any research has not yet solved the questions as to how the appearance of the body-fat controlling function of the amount of body fat in terms of the content of medium chain triglyceride in an agent, the kinds and proportions of fatty acid residues constituting the medium chain triglyceride, or the bonding positions of the fatty acid residues.

Japanese Patent Unexamined Publication (JP Kokai) 2000-309794 discloses an oil and fat composition comprising triglycerides, wherein medium chain fatty acids is present in an amount of 5 to 23% by mass in the total fatty acids and triglycerides having two medium chain fatty acid residues in the molecule thereof constitute 1 to 20% by mass of the total triglycerides. However, there is not disclosed any regulator for the amount of body fat comprising medium chain triglyceride as the main component.

### DISCLOSURE OF THE INVENTION

An object of the present invention is to provide a regulator for the amount of body fat that can be used conveniently without any need for food restriction and any concern about the side effect of the regulator for the amount of body fat itself.

Another object of the present invention is to provide a food product for regulating the amount of body fat comprising the above-mentioned regulator for the amount of body fat.

The present invention has been accomplished based on the findings that medium chain triglyceride itself has the function of controlling the amount of body fat to the ideal level.

Accordingly, the present invention provides a regulator for the amount of body fat comprising a medium chain triglyceride, wherein 90% by mass or more of fatty acids constituting the medium chain triglycerides are comprised of saturated fatty acids having 8 and 10 carbon atoms, the ratio by mass of the saturated fatty acids having 8 carbon atoms to the saturated fatty acids having 10 carbon atoms is 60:40 to 85:15, and the saturated fatty acids having 8 carbon atoms is present in an amount of 60 to 85% by mass of the total fatty acids bonded to the triglycerides at the 2-position.

Further, the present invention provides a regulator for the amount of body fat comprising a medium chain triglyceride in an amount of 40% by mass or more.

The present invention also provides use of the above-mentioned regulator for the amount of body fat for controlling the accumulation of the body fat of those having a BMI value of 23 or more.

In addition, the present invention provides a food product for regulating the amount of body fat comprising the above-mentioned regulator for the amount of body fat.

### BEST MODE FOR CARRYING OUT THE INVENTION

The medium chain triglyceride used in the present invention is a triglyceride having as the constituent fatty acids medium chain fatty acids, which is also referred to as triacylglycerol. The medium chain fatty acids used in the present invention include fatty acids having 6 to 12 carbon atoms, preferably saturated fatty acids, and more preferably saturated fatty acids having an even number of carbon atoms. For example, the medium chain fatty acids used in the present invention include caproic acid, caprylic acid, capric acid, and lauric acid. In particular, preferably used are caprylic acid and capric acid, which are saturated fatty acids having 8 and 10 carbon atoms respectively. In the regulator for the amount of body fat of the present invention, 90% by mass or more, preferably 95 to 100% of fatty acids constituting the medium chain triglycerides are comprised of saturated fatty acids having 8 and 10 carbon atoms, the ratio by mass of the saturated fatty acids having 8 carbon atoms to the saturated fatty acids having 10 carbon atoms is 60:40 to 85:15, preferably 70:30 to 80:20, and the saturated fatty acids having 8 carbon atoms is present in an amount of 60 to 85% by mass, preferably 65 to 80% by mass, of the total fatty acids bonded to the triglycerides at the 2-position.

The method for producing the above-mentioned medium chain triglyceride is not particularly limited. For example, medium chain fatty acids derived from palm kernel oil and coconut oil are used with glycerol as the raw materials, which may be subjected to an ester linkage reaction to obtain the desired medium chain triglyceride. There is no limitation as to the conditions of the ester bond reaction. For example, the reaction may be carried out under application of pressure in the absence of any catalyst and any solvent. As a matter of course, the medium chain triglyceride used in the present invention can be obtained through the reaction using some catalysts and solvents. The medium chain triglyceride used in the present invention can be obtained by mixing a medium chain triglyceride and a medium chain fatty acid at a controlled mixing ratio, and conducting the reaction therebetween using a lipase having resiospecificity, for example, “Lipase PL” commercially available from Meito Sangyo Co., Ltd. Namely, the medium chain triglyceride can thus be provided in such a structure that the constituent fatty acids comprises saturated fatty acids having 8 and 10 carbon atoms in an amount of not less than 90% by mass, the ratio by mass of the saturated fatty acids having 8 carbon atoms to the saturated fatty acids having 10 carbon atoms is 60:40 to 85:15, and the saturated fatty acids having 8 carbon atoms is present in an amount of 60 to 85% by mass of the total fatty acids bonded to the triglycerides at the 2-position. Further, the medium chain triglyceride itself can be obtained from seeds of genetically modified plant from which oil is extracted. Alternatively, the medium chain triglyceride can also be prepared by using the medium chain fatty acids obtained from such genetically modified plant seeds as the raw materials.

In the context of the present invention, to regulate or control the body fat means the function of regulating or controlling the amount of body fat, by decreasing the amount of body fat when the body fat is excessively accumulated, having no effect on the amount of body fat when the amount of body fat is within the proper range, and increasing the amount of body fat when the amount of body fat is insufficient.

It is preferable that the regulator for the amount of body fat of the present invention comprise the above-mentioned medium chain triglyceride in an amount of 5% by mass or more, more preferably 5 to 100% by mass, and further preferably 40 to 99.95% by mass.

The present invention also provides a regulator for the amount of body fat comprising medium chain triglycerides generally used in an amount of 40% by mass or more. In this type of regulator for the amount of body fat, it is preferable that the amount of the medium chain triglycerides be in the range of 40 to 99.95% by mass, and more preferably in the range of 48 to 99.95% by mass.

The regulator for the amount of body fat according to the present invention may further comprise other components together with the medium chain triglyceride. Such additive components are not particularly limited, but preferably include typical edible oils such as soyabean oil, rapeseed oil, corn oil, sesame oil, sesame salad oil, perilla oil, linseed oil, peanut oil, safflower oil, high oleic acid safflower oil, sunflower oil, high oleic acid sunflower oil, cottonseed oil, grape seed oil, macadamia ternifolia seed oil, hazelnut oil, pumpkin seed oil, walnut oil, camellia oil, tea oil, borage oil, olive oil, rice bran oil, wheat germ oil, palm oil, palm kernel oil, coconut oil, cocoa butter, beef tallow, lard, chicken oil, dairy cream, fish oil, seal oil and algae oil, and modified oils of the above-mentioned edible oils, such as low saturated oils by breeding, hydrogenated oils thereof, fractioned oils and the like. Also, the regulator for the amount of body fat of the present invention may further comprise not only vitamin E and phytosterol, but also polyglycerol fatty acid esters, sucrose fatty acid esters, sorbitan fatty acid esters, fatty acid esters of ascorbic acid, lignan, coenzyme Q, phospholipids, oryzanol, diglycerides, and the like.

In particular, it is preferable that the regulator for the amount of body fat of the present invention comprise at least one component selected from the group consisting of animal and vegetable oils, vitamin E, and phytosterol. When the animal and vegetable oil is added, the animal and vegetable oil may preferably be contained in an amount of 60% by mass or less, more preferably in an amount of 5 to 55% by mass, and further preferably in an amount of 5 to 50% by mass of the total mass of the regulator. In the case where vitamin E is added, the vitamin E may preferably be contained in an amount of 0.01 to 5% by mass, more preferably 0.01 to 2% by mass of the total mass of the regulator. When the phytosterol is added, the phytosterol may preferably be contained in an amount of 0.5 to 6% by mass, more preferably 1 to 4% by mass of the total mass of the regulator.

The regulator for the amount of body fat of the present invention may be formed into any shape. The regulator for the amount of body fat may be supplied in a capsule form and a tablet form or the like. The regulator for the amount of body fat may be used by addition to food products, processed food products, beverages, flavoring materials and confection, for example, gelatin capsule, edible oil, dressing, margarine, prepared margarine, fat spread, cream, ice cream, mayonnaise, bread, cake, doughnut, muffin, scone, deep-fried food, snack food, liquid diet and so on.

The present invention can thus provide the regulator for the amount of body fat and the food products for regulating the amount of body fat comprising the same, which are capable of regulating the amount of body fat with ease, safety and high effectiveness.

For instance, the amount of body fat of a person can be controlled to about 21.5 to 22.5, especially to about 22 in terms of the body mass index (BMI) by using the regulator for the amount of body fat of the present invention. The term BMI, which is an indicator used to make a medical assessment of obesity, is described in detail in “Himansho: Shindan, Chiryo, Shido no Tebiki [Obesity Handbook: Diagnosis, Treatment and Instruction]” Japan Society for the Study of Obesity, editorial board for a diagnosis handbook for obesity, pp. 14-24, Ishiyaku Publishers, Inc. 1993. To measure the amount of body fat, there are a variety of methods, for example, total body densitometry including an underwater weighing method and air displacement method, internal K⁴⁰ counting method, bioelectrical impedance analysis method, and bone density measuring method. The measurement of the body fat requires much time and various facilities according to the above methods, so that the assessment by the BMI which is obtained based on the data of the height and the weight is usually employed. The BMI is determined from the formula of [weight (kg)/height (m)/height (m)], and the lower the value of the BMI, the leaner the person is and the less the body fat is; while the higher the value of the BMI, the more body fat the person has. The BMI of 22 is the most ideal value.

The present invention will be hereinafter illustrated with reference to the following Examples, but the present invention is not limited to those specific Examples.

### EXAMPLE 1

A regulator for the amount of body fat No. 1 was obtained by mixing 500 g of a medium chain triglyceride commercially available from The Nisshin Oil Mills Ltd. under the trade name of ODO with 500 g of a rapeseed oil commercially available from The Nisshin Oil Mills Ltd. A regulator for the amount of body fat No. 2 was obtained by mixing 900 g of a medium chain triglyceride commercially available from The Nisshin Oil Mills Ltd. under the trade name of ODO with 100 g of lard produced by Snow Brand Milk Products Co., Ltd. In the above-mentioned medium chain triglyceride, saturated fatty acids having 8 and 10 carbon atoms was present in an amount of 99.9% by mass of the total constituent fatty acids, with the ratio by mass of the saturated fatty acids having 8 carbon atoms to the saturated fatty acids having 10 carbon atoms being 7.4:2.6. The saturated fatty acids having 8 carbon atoms was present in an amount of 73% by mass of all the fatty acids bonded to the triglycerides at the 2-position. The fatty acid in the 2-position of the triglyceride was determined by Brockerhoff method (Brockerhoff, H., Journal of Lipid Research, Vol. 6, p.10, 1965).

### EXAMPLE 2

A regulator for the amount of body fat No. 3 was obtained by adding 1 g of a medium chain tocopherol commercially available from The Nisshin Oil Mills Ltd. under the trade name of Tocopherol 85 to 999 g of a medium chain triglyceride commercially available from The Nisshin Oil Mills Ltd. under the trade name of ODO, and thoroughly stirring the resultant mixture to dissolve the tocopherol in the triglyceride. In the above-mentioned medium chain triglyceride, saturated fatty acids having 8 and 10 carbon atoms was present in an amount of 99.9% by mass of the total constituent fatty acids, with the ratio by mass of the saturated fatty acids having 8 carbon atoms to the saturated fatty acids having 10 carbon atoms being 7.4:2.6 The saturated fatty acids having 8 carbon atoms was present in an amount of 73% by mass of all the fatty acids bonded to the triglycerides at the 2-position.

### EXAMPLE 3

A regulator for the amount of body fat No. 4 was obtained by adding 20 g of a phytosterol derived from rice oil to 980 g of the regulator for the amount of body fat No. 1 obtained in Example 1, and thoroughly mixing and stirring the resultant mixture.

### EXAMPLE 4

Using the regulator for the amount of body fat No. 3 obtained in Example 2, rolls (bread) were prepared by the following procedures. Those rolls are referred to as a food product for regulating the amount of body fat No. 1. Each roll contained 5 g of the regulator for the amount of body fat No. 3.

<Formulation for rolls>

| | Parts by Mass |
|---|---|
| Strong flour | 90 |
| Weak flour | 10 |
| Sugar | 12 |
| Salt | 1.7 |
| Whole egg | 15 |
| Skimmed milk powder | 4 |
| Yeast | 4 |
| Emulsifier | 0.5 |
| Body fat regulator | 24 |
| Water | 41 |

<Manufacturing Procedure>

Preparation: straight dough method

Specified final dough temperature: 28° C.

Fermentation time: 60 minutes

Dividing: 42 g

Bench time: 20 minutes

Proof: 50 minutes at 37° C.

Baking: 10 minutes at 205° C.

### EXAMPLE 5

Using the food product for regulating the amount of body fat No. 1 obtained in Example 4, a test was conducted to evaluate the regulating effects for the amount of body fat. Test subjects consisting of 78 test subjects were divided into two groups. One group was required to eat two rolls according to the present invention (the food product for regulating the amount of body fat No. 1) every morning over a period of 12 weeks. Comparative rolls which were prepared in the same manner as in Example 4 except that the regulator for the amount of body fat No. 3 was replaced by a prepared salad oil were supplied to the other group, that is, a control group. All the test subjects were instructed to have a diet during the test period so that the daily total calories supplied might be 2200 kilocalories and the daily total intake of lipid might be 60 g. The weight, body fat, waist size, and visceral fat of each person were measured before the test, 8 weeks after completion of the test, and 12 weeks after completion of the test. The body fat was determined using a commercially available measuring instrument (“MAB-1000”, made by Nihon Kohden Corporation), and the visceral fat mass was obtained from the visceral fat area on a CT-scan slice of the abdomen of each subject. The test results are shown in Table 1. Statistical analysis was performed by classifying the subjects of each group into two categories, the one with a BMI of 23 or more and the other with a BMI of less than 23.

TABLE 1
Test Results about Body Fat Regulator Containing Food
Product No. 1 (Changes between before and after the test)

| | | | Food of Present | |
|---|---|---|---|---|
| | Comparative Food | | Invention | |
| | BMI ≧ 23 | BMI < 23 | BMI ≧ 23 | BMI < 23 |
| Weight (kg) | | | | |
| After 8 w. | −3.36 ± 0.3 | −2.13 ± 0.6 | −4.50 ± 0.4* | −2.21 ± 0.4 |
| After 12 w. | −4.78 ± 0.4 | −3.08 ± 0.7 | −6.12 ± 0.5* | −3.30 ± 0.6 |
| Body fat | | | | |
| amt. (kg) | | | | |
| After 8 w. | −2.75 ± 0.2 | −2.02 ± 0.4 | −3.86 ± 0.3* | −1.72 ± 0.4 |
| After 12 w. | −3.61 ± 0.4 | −2.49 ± 0.5 | −4.57 ± 0.5 | −2.36 ± 0.5 |
| Waist (cm) | | | | |
| After 8 w. | −2.26 ± 0.5 | −1.19 ± 0.6 | −3.05 ± 0.4 | −1.80 ± 0.5 |
| After 12 w. | −3.74 ± 0.5 | −2.40 ± 0.6 | −5.67 ± 0.5* | −3.71 ± 0.7 |
| Visceral | | | | |
| fat (cm) | | | | |
| After 8 w. | −18.9 ± 2.5 | −12.6 ± 4.2 | −31.6 ± 3.0* | −14.9 ± 2.9 |
| After 12 w. | −32.6 ± 0.5 | −26.0 ± 4.2 | −48.8 ± 5.1* | −19.9 ± 3.7 |

*There is a statistical significance. (risk rate: 5% or less)

As shown in Table 1, there are significant decreases in the weight, body fat, waist size, and visceral fat area of the subjects with a BMI of 23 or more who took the food product containing the regulator for the amount of body fat according to the present invention. It becomes evident from the above results that the body fat regulating food product No. 1 that contains the regulator for the amount of body fat according to the present invention can work to regulate the accumulation of the body fat mass with respect to the persons with a BMI of 23 or more.

What is claimed is:

1. A regulator for the amount of body fat comprising a medium chain triglyceride, wherein 90% by mass or more of fatty acids constituting medium chain triglycerides are comprised of saturated fatty acids having 8 and 10 carbon atoms, the ratio by mass of the saturated fatty acids having 8 carbon atoms to the saturated fatty acids having 10 carbon atoms is 60:40 to 85:15, and the saturated fatty acids having 8 carbon atoms is present in an amount of 60 to 85% by mass of the total fatty acids bonded to the triglycerides at the 2-position.

2. The regulator for the amount of body fat as claimed in claim 1, wherein the medium chain triglyceride is contained in an amount of 5% by mass or more.

3. A regulator for the amount of body fat comprising a medium chain triglyceride in an amount of 40% by mass or more.

4. The regulator for the amount of body fat as claimed in any one of claims 1 to 3, further comprising an animal and vegetable oil.

5. The regulator for the amount of body fat as claimed in any one of claims 1 to 4, further comprising vitamin E.

6. The regulator for the amount of body fat as claimed in any one of claims 1 to 5, further comprising a phytosterol.

7. A method for regulating the amount of body fat comprising the step of administrating the regulator for the amount of body fat as claimed in any one of claims 1 to 6 to those having a BMI value of 23 or more and controlling the accumulation of body fat of them.

8. A food product for regulating the amount of body fat, comprising the regulator for the amount of body fat as claimed in any one of claims 1 to 6.

9. The food product for regulating the amount of body fat as claimed in claim 8, which is in the form of an edible oil, mayonnaise, margarine, prepared margarine, fat spread, dressing, bread, ice cream, cream, confection, doughnut, muffin, scone, or deep-fried food.
